(19) United States
(12) Patent Application Publication
(10) Pub. No.: US 2017/0314047 A1
(43) Pub. Date: Nov. 2, 2017

(54) METHOD FOR THE PRODUCTION OF ALKYL ESTERS

(71) Applicant: Viesel Skunk Works, LLC, Stuart, FL (US)

(72) Inventors: Stuart LAMB, Stuart, FL (US); Franklin MATHIS, Dublin, GA (US); Brent CHRABAS, Stuart, FL (US)

(21) Appl. No.: 15/528,995

(22) PCT Filed: May 19, 2015
(86) PCT No.: PCT/US2015/031570

§ 371 Date: May 23, 2017

Related U.S. Application Data
(60) Provisional application No. 62/085,964, filed on Dec. 1, 2014.

(51) Int. Cl. C12P 7/64; C07C 67/54; C10L 1/02

(52) U.S. Cl. CPC C12P 7/649; C10L 1/026; C07C 67/54; C12Y 301/01003; C10L 2200/0476; C10L 2270/026; C10L 2290/542; C10L 2290/543; C10L 2290/26

(57) ABSTRACT

A method of producing biodiesel includes reacting at least one of brown grease and FOG with at least one enzyme, alcohol, and an aqueous solution to produce a reacted feedstock and producing biodiesel from the reacted feedstock, the biodiesel having a composition of sulfur less than 15 ppm.

The present invention relates to a method and system for producing alkyl esters, and more particularly, to a method for producing alkyl esters from feedstock containing high free fatty acids and high sulfur levels.

### BACKGROUND OF THE INVENTION

Biodiesel, the mixture of mono-alkyl esters of long chain fatty acids produced from either through a transesterification reaction between the triglycerides in plant oils or animal fats with methanol or an esterification reaction between free fatty acids (FFAs) and methanol, is a low-emission diesel substitute fuel and can be used as in its pure form or blended with petroleum diesel. Compared with petroleum diesel, biodiesel is safe, renewable, non-toxic, and biodegradable. Its usage also generates numerous societal benefits, such as rural revitalization, creation of new jobs, and reduced global warming. A wide range of processes have been investigated for biodiesel production, but the base-catalyzed production process is the predominant one to be successful for commercial implementation at industrial scale of production, which requires the use of high quality, high purity virgin oils.

The predominant production mode of the base-catalyzed process is a batch or semi-continuous process (reactants added continuously to a flow reactor), which results in low yield, large variation in product quality, and intensive labor and energy requirements. Operational problems in the conventional production process are typically linked to the catalyst (e.g. potassium and sodium hydroxide) because they are hazardous, caustic, and hygroscopic.

While there are advantages of biodiesel over the traditional petroleum based diesel, biodiesel commercialization is limited by production cost that is dominated by the price of the feedstock. However, the chemistry of the base transesterification reaction in use limits feedstock flexibility due to unwanted side reactions (neutralization reactions). Depending upon cultivation conditions and its availability at different geographic regions, more than 95% of total biodiesel is currently oil produced from edible oil feedstock; thus, its competition with food consumption has been a global concern. Edible oils such as rapeseed oil (84%) and sunflower roil (13%) are the major contributor as feedstock in biodiesel production followed by palm oil (1%) and the remaining from soybean, groundnut, coconut, peanut, corn and canola (2%). These feedstocks are high cost, which currently accounts for over 85% of biodiesel production expenses. In order to minimizing the feedstock cost, food competition and environmental issues, fats, oil and grease (FOG) recovered from restaurants, food processing plants and grease interceptors in wastewater plants, usually have also been explored for their utility as feedstocks for biodiesel production. However, none of the traditional processes is successful with converting FOG into biodiesel economically due to its high free fatty acid (FFA) content and large quantities of contaminants. One of the key impurities unique to interceptor grease, is the high sulfur content up, which can have concentrations of up to 10,000 ppm and which would need to be reduced to 15 ppm or less in the finished biodiesel product to meet government standards for use.

### SUMMARY OF THE INVENTION

The present invention advantageously provides a method and system for the production of alkyl esters, the method includes pretreating a feedstock including a mixture of glycerides, free fatty acids, and sulfur to remove water and solids to create a pretreated feedstock. The pretreated feedstock and at least one of an aqueous solution and water is introduced into at least one continuous stir tank reactor. At least one enzyme and alcohol is introduced into the at least one continuous stir tank reactor to elicit an enzyme catalyzed reaction with the pretreated feedstock, the enzyme catalyzed reaction creating reacted contents. The reacted contents exiting the at least one continuous stir tank reactor are separated into a glycerin phase and a crude alkyl ester phase. The crude alkyl ester phase is distilled to remove sulfur to provide a polished alkyl ester phase. The polished alkyl ester phase is passed through a cat-ion exchange apparatus to produce refined alkyl esters with the reduced FFA level.

In another embodiment, the method includes pretreating a feedstock including a mixture of glycerides, free fatty acids, and sulfur to remove water and solids to create a pretreated feedstock. The pretreated feedstock and at least one of water and a caustic aqueous solution is introduced into a one of a plurality of continuous stir tank reactors arranged in series, the plurality of continuous stir tank reactors being in fluid communication with each other. At least one enzyme and alcohol is introduced into each of the plurality continuous stir tank reactors to elicit an enzyme catalyzed reaction with the pretreated feedstock, the enzyme catalyzed reaction creating reacted contents. The reacted contents exiting the last one in series of the plurality of continuous stir tank reactors are separated into a glycerin phase and a crude alkyl ester phase. The crude alkyl ester phase is passed through a cat-ion exchange apparatus having a plurality of a resin beds to produce refined alkyl esters. The refined alkyl ester phase is introduced into a polar adsorptive media to produce polished alkyl esters. The polished alkyl esters are treated with an antioxidant.

In yet another embodiment, the method includes pretreating a feedstock having a free fatty acid composition of at least 10% by dry weight and at least 40 parts per million sulfur to remove water and solids creating a pretreated feedstock. The pretreated feedstock and at least one of water and an aqueous solution is introduced into a one of a plurality of continuous stir tank reactors arranged in series, the plurality of continuous stir tank reactors being in fluid communication with each other. At least one enzyme and alcohol is introduced into each of the plurality of continuous stir tank reactors to elicit an enzyme catalyzed reaction with the pretreated feedstock, the enzyme catalyzed reaction creating reacted contents. The reacted contents exiting the at least one continuous stir tank reactor are separated into a glycerin phase and a crude alkyl ester phase. The crude alkyl ester phase is passed through a stripping column in fluid communication with a reboiler to distill the crude alkyl ester phase at a first temperature and to create still bottoms. At least a portion of the still bottoms are diverted into a main still for removal to distill the still bottoms at a second temperature higher than the first temperature to create a polished alkyl ester phase. The polished alkyl ester phase is passed through a plurality of resin beds and introducing alcohol into the plurality of resin beds when the polished alkyl ester phase is passed through the plurality of resin beds to produce refined alkyl esters. The refined alkyl esters are treated with an antioxidant.

In yet another embodiment, the method includes reacting at least one of brown grease and FOG with at least one enzyme, alcohol, and an aqueous solution to produce a reacted feedstock and producing biodiesel from the reacted feedstock, the biodiesel having a composition of sulfur less than 15 ppm.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a process flow diagram of an exemplary alkyl ester production system for high sulfur feedstocks constructed in accordance with the principles of the present application;

FIG. 2 is a flow chart of an exemplary alkyl ester production method for high sulfur feedstocks constructed in accordance with the principles of the present application;

FIG. 3 is a process flow diagram of an exemplary alkyl ester production system for low sulfur feedstocks constructed in accordance with the principles of the present application;

FIG. 4 is a flow chart of an exemplary alkyl ester production method for high sulfur feedstocks constructed in accordance with the principles of the present application;

FIG. 5 is flow chart of an exemplary method of producing a refined feedstock from crude feedstock used in both of the methods shown in FIGS. 2 and 4;

FIG. 6 is a flow chart of exemplary method of producing crude alkyl esters from a refined feedstock used in both of the methods shown in FIGS. 2 and 4;

FIG. 7 is a process flow diagram of an exemplary crude ester alky ester system;

FIG. 8 is a flow chart of an exemplary method of producing a refined biodiesel from crude alkyl esters used in the method shown in FIG. 2;

FIG. 9 is a flow chart of another exemplary method of producing a refined biodiesel from crude alkyl esters;

FIG. 10 is a flow chart of an exemplary method of producing a refined biodiesel from crude alkyl esters used in the method shown in FIG. 4; and

FIG. 11 is a chart showing the initial and final bound glycerin by dry weight percentage, free fatty acid by dry wait percentage, and sulfur in parts per million for different feedstocks.

### DETAILED DESCRIPTION OF THE INVENTION

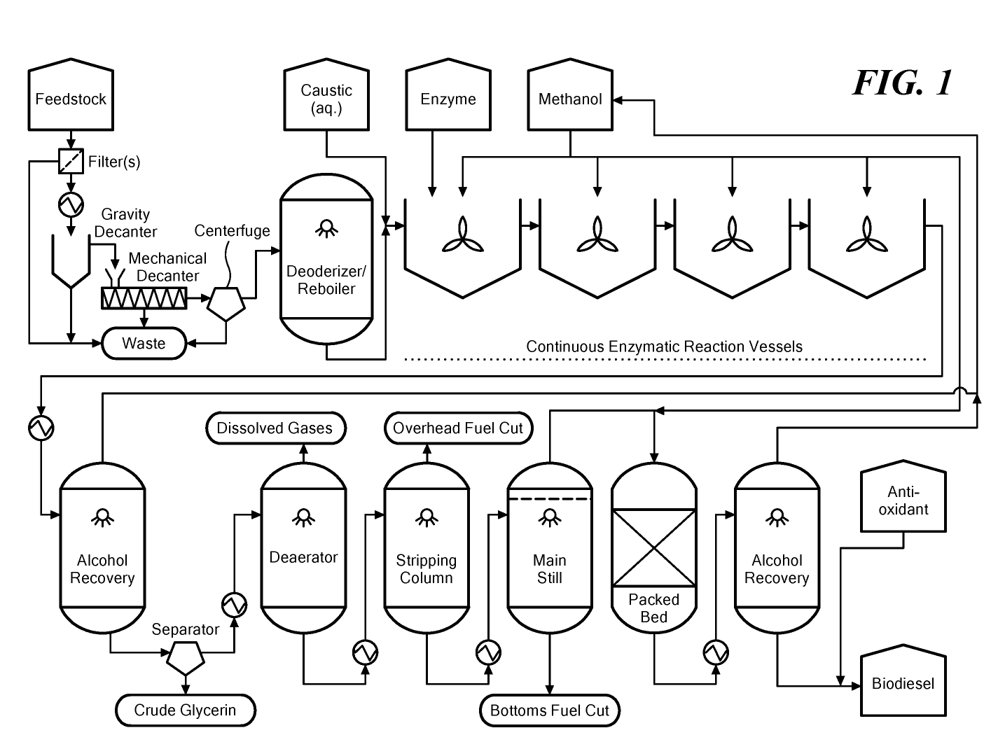

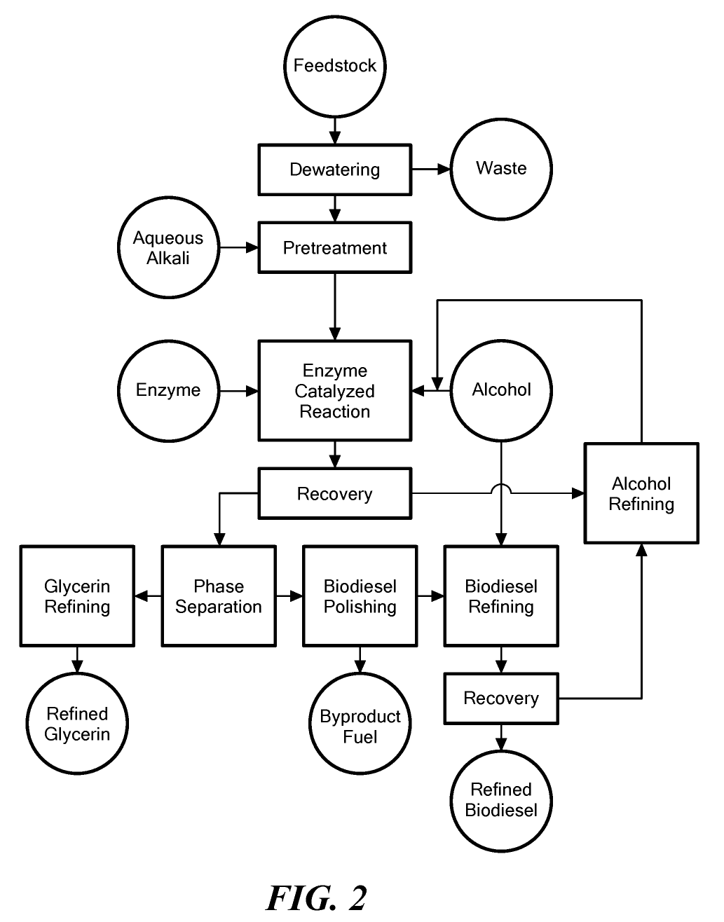

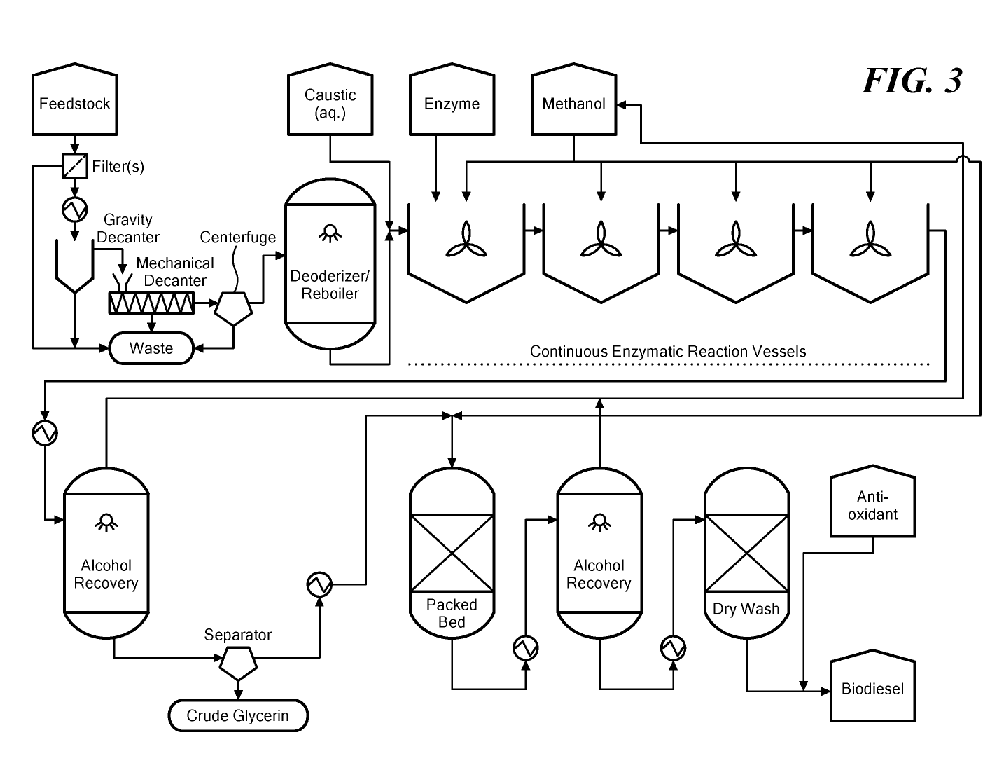

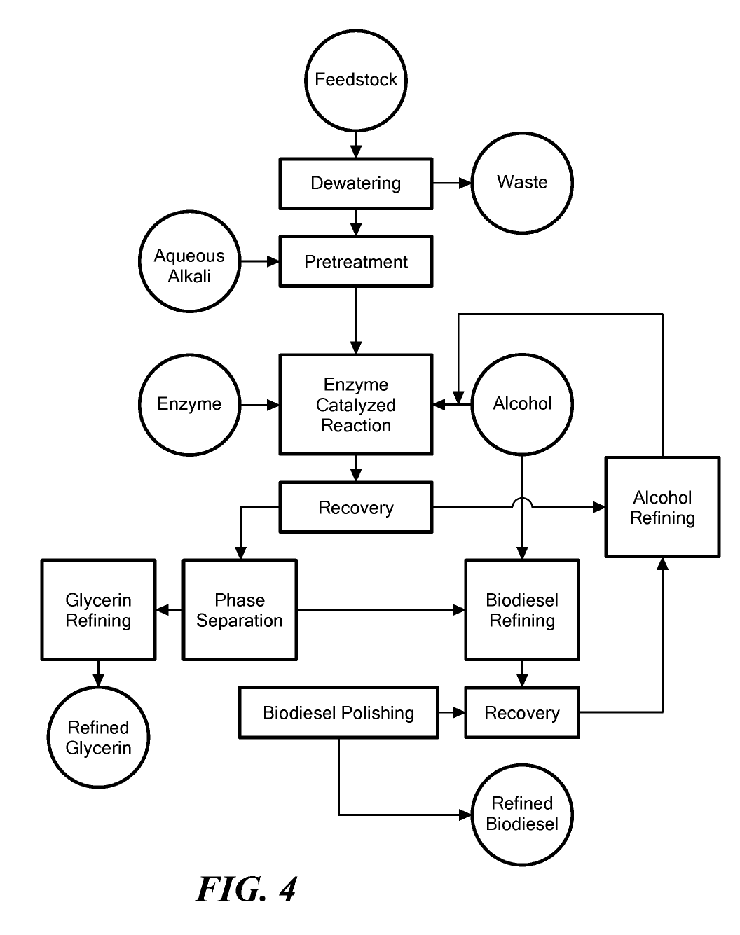

Referring now to the drawings in which like reference designators refer to like elements, there is shown in FIGS. 1-4 an exemplary system and method for producing alkyl esters from high sulfur (FIGS. 1-2) and low sulfur (FIGS. 3-4) feedstocks designated generally as “10.” The term feedstock as used herein refers to waste oils such as, but not limited to, yellow grease, brown grease, municipal fats, oils, and greases (FOG), lard, tallow, orange oil, fish oil, carinata oil, corn oil, palm fatty acid distillate, or any used cooking oil having a mixture of triglycerides (long and/or short chain), free fatty acids, and sulfur. In an exemplary configuration, the feedstock is composed of FOG having at least 10% free fatty acid concentration by dry weight and at least 300 ppm of sulfur or higher. In other configurations, the free fatty acid concentration in the feedstock may be as low as 0.5% and sulfur levels below anywhere between 5-40 ppm sulfur. The terms low and high sulfur feedstocks are relative. In an exemplary configuration, a low sulfur feedstock refers to feedstock with lower than 40 ppm sulfur and any concentration of sulfur higher than 40 ppm sulfur may be referred to as a high sulfur feedstock.

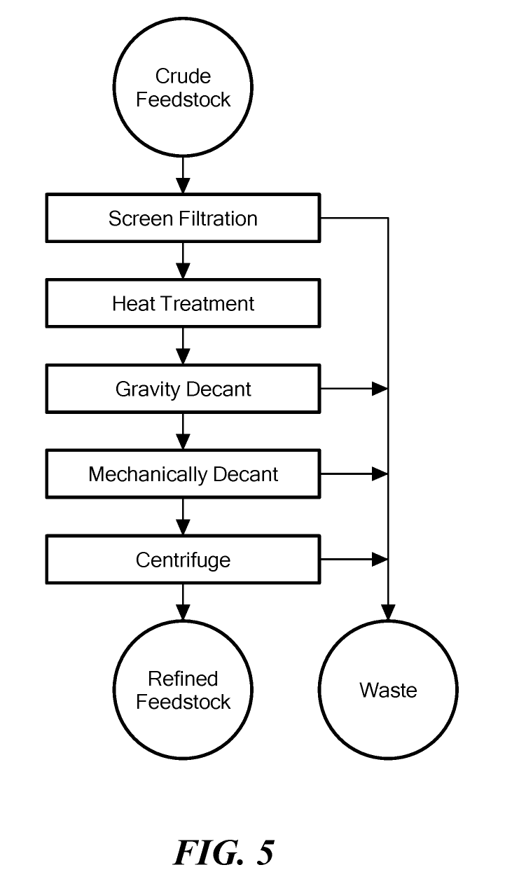

In both the high sulfur and low sulfur feedstock, the feedstock may be dewatered before it is pretreated. For example, exemplary crude feedstocks may contain up to 95% water and unsaponifiable material that may be removed to recover the oils for conversion to alkyl esters. As shown in FIG. 5, one example of a dewatering process may include initially passing the feedstock through a screen filter to remove large particles, for example, food particles that may be present in certain waste oils. The feedstock may further be heated to approximately 135-220 degrees Fahrenheit to kill off any pathogens and to reduce the viscosity of the feedstock. A gravity decanter may be utilized to remove water and smaller non-oil particles. To remove the unsaponifiable material, the feedstock may then be passed through a mechanical decanter and a centrifuge to remove impurities. Each of the above dewatering steps may be done in series.

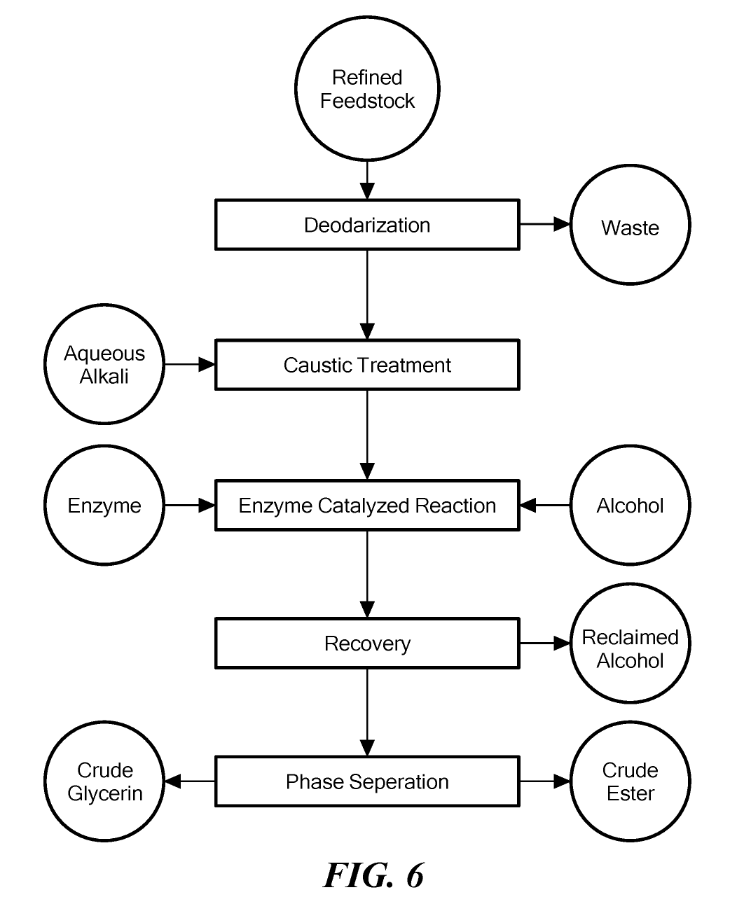

Referring now to FIG. 6, the dewatered or refined feedstock, which in an exemplary configuration may contain approximately 0.5% by weight volatile impurities, may be further processed to create a pretreated feedstock. In particular, the dewatered or refined feedstock may be passed through a reboiler operating at approximately 135-220 degrees Fahrenheit under a vacuum to remove remaining volatile impurities and dissolved gases. At least a portion of the bottoms of the reboiler may be combined with an aqueous alkaline solution introduced at a treatment rate of approximately 25-250 ppm. For example, a caustic aqueous solution, for example, a sodium hydroxide aqueous solution, and/or water, may be combined with the bottoms of the reboiler to neutralize mineral acids that may be present in the feedstock stream exiting the reboiler to create a pretreated feedstock.

The pretreated feedstock may then be pumped or otherwise directed into one or more continuous stir tank reactors (“CSTR”). In the configuration shown in FIG. 1, four CSTRs are arranged in series. In other configurations, the one or more CSTRs may be arranged in parallel. In an exemplary configuration, the pretreated feedstock may be introduced into the first CSTR, or substantially simultaneously into each of the CSTRs, at a rate of 0.5-1 gal/min. Additionally, 3-10 ml/min of at least one enzyme may be introduced into the first CSTR or substantially simultaneous into each of the CSTRs. The enzyme may be a lipase containing Candida Antarcitca Lipase B and is configured to catalyze the CSTR components and convert the glycerides and FFA to esters. Water may be introduced into the first CSTR or substantially simultaneously into each of the CSTRs at a rate of 20-60 ml/min and an alcohol, such as methanol may be introduced in each of the CSTRs at a rate of 200-600 ml/min. The mass flow rates of feedstock, enzyme, water, and methanol may be constant, variable, and may be adjustable automatically or manually. In an exemplary configuration, the temperature of the reaction in the CSTRs is approximately 85-115 degrees Fahrenheit and may be carried out at a pressure of between 0-5 psig.

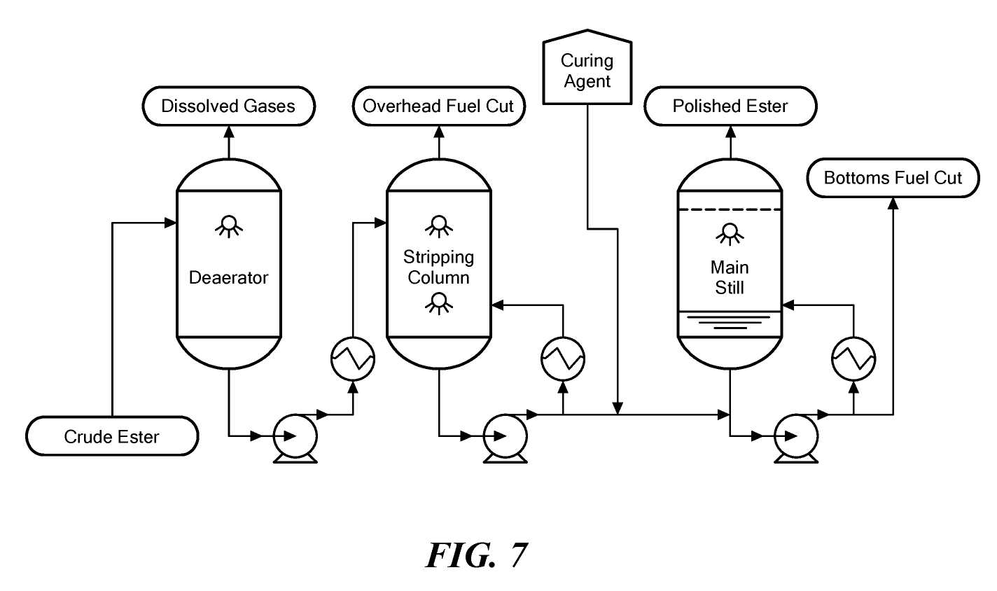

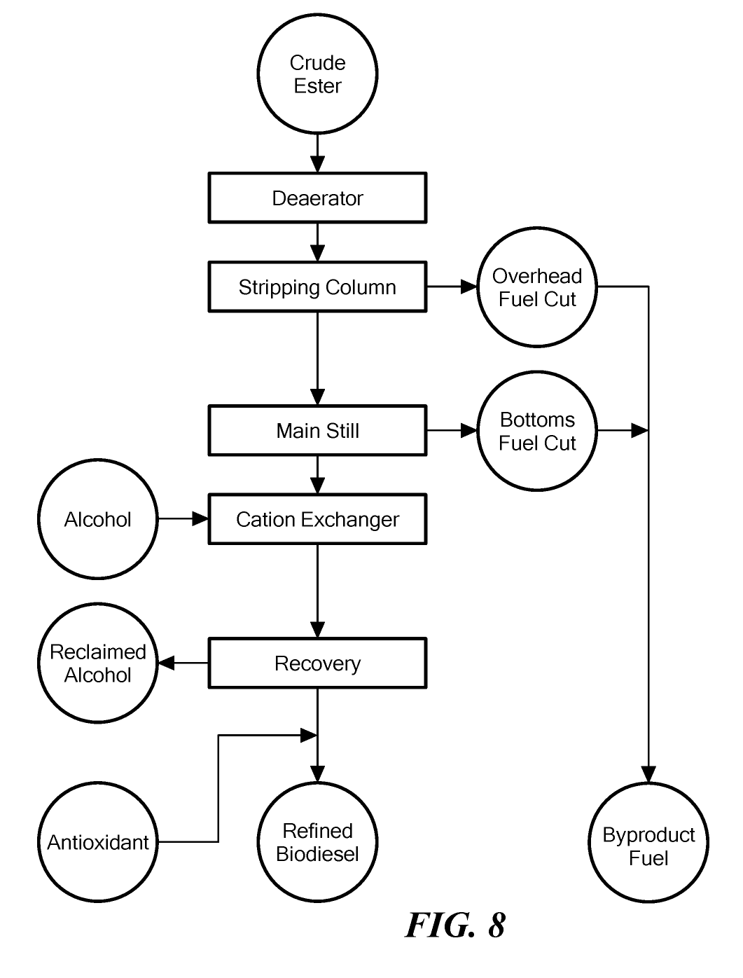

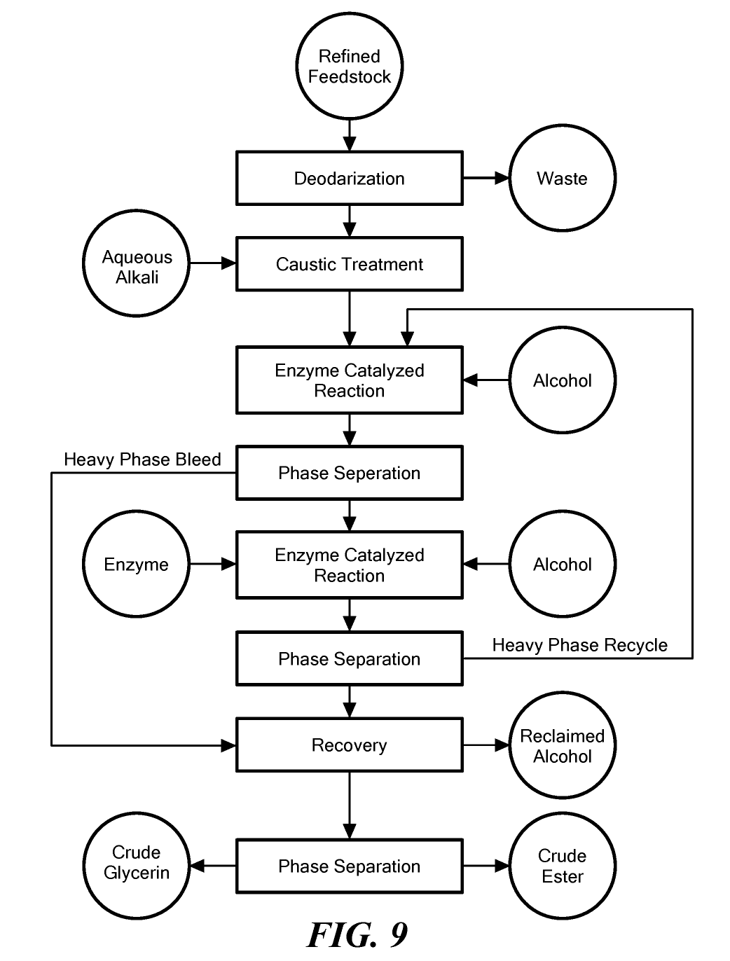

Referring now to FIGS. 7 and 8, the outflow from the CSTRs, namely, the reacted pretreated feedstock exiting either the last CSTR in series, or all of the CSTRs if arranged in parallel, comprises a stream of crude alkyl esters and a glycerin phase. In an exemplary configuration, the reacted contents exiting the CSTRs may include approximately 3-10% free fatty acids, bound glycerin, free glycerin, water, and excess alcohol. The reacted contents may further be processed to further separate the stream components and to recycle the alcohol for reintroduction into the enzyme catalyzed reaction. In particular, volatiles within the reacted contents stream may be removed under vacuum, for example, at a pressure between −14-0 psig, for further refining. The ester phase and the glycerin phase may further be separated by use of, for example, a continuous oil coalescing system, which removes the glycerin from the system for further refining and created a crude alkyl ester phase. In an alternative configuration, as shown in FIG. 9, the reacted contents may be separated in stages and in series with the enzyme catalyzed reaction to allow for the recycling of enzyme. For example, phase separation may occur between each CSTR catalyzed reaction. For example, after the first the enzyme catalyzed reaction occurs in the first CSTR, the glycerin may be at least partially removed from the reacted contents stream to recover alcohol and after the enzyme catalyzed reaction occurs in a downstream CSTR, the enzyme now present in the glycerin phase may be recycled to be re-used in the first CSTR.

Referring back now to FIGS. 7-8, the crude alkyl ester phase may then enter a polishing phase in configurations in which a high sulfur feedstock, for example, FOG, is used as the feedstock. In particular, when the crude alkyl esters include up to 15% free fatty acid, 2% bound glyceride and up to 10,000 ppm sulfur, the crude alkyl esters may enter a polishing phase to remove sulfur. It is understood that the above concentrations of free fatty acid, bound glyceride and sulfur are merely exemplary, and the crude feedstock may enter the polishing phase independent of the concentration of any of those components. The crude alkyl ester may be pumped or otherwise introduced into in a flash deaerator to remove dissolved gasses. In an exemplary configuration, the crude alkyl ester is preheated to approximately 300 degrees Fahrenheit before entering the flash deaerator. The bottoms of the deaerator are then pumped and heated to approximately 375 degrees Fahrenheit before entering a stripping column and a reboiler loop to remove sulfur. The stripping column/reboiler loop is configured to operate under a vacuum of approximately −14 psig and to remove approximately 90% of the dissolved sulfur and light organic sulfur compounds in the crude alkyl ester stream. The distillate overhead fuel may be removed from the system for further use. At least a portion of the bottoms of the stripped alkyl ester stream may further be combined with a curing agent such as but not limited to initiators, accelerators, or promoters before being introduced into a main still where the temperature in the main still is raised to temperature higher than the temperature in the stripping column. For example, the temperature in the main still may be raised to approximately 400-500 degrees Fahrenheit and may operate under a vacuum of approximately −14 psig. The main still operates to further remove approximately 90% of the sulfur remaining in the stripped alkyl ester stream. In an exemplary configuration, the main still operates for up to 24 hours to create still bottoms containing heavier organic and inorganic sulfur compounds. In particular, the curing agent crosses linking of polymers with the sulfur through vulcanization, which binds the remaining sulfur. The distillate leaving the main still is a polished alkyl ester and the bottoms may be removed from the system. The temperatures and pressures discussed above in the polishing phase are merely exemplary, and it is contemplated that the temperatures may range from 150-500 Fahrenheit in the polishing phase and the pressure may range from −14-0 psig.

Now referring back now to FIG. 1-2, the polished alkyl ester may then be refined to produce biodiesel. In particular, following the polished alkyl ester phase, the distillate from the main still may be combined with a stream of alcohol, for example, methanol before entering a cat-ion exchanger. The cat-ion exchanger may include a plurality of resin beds arranged in series or in parallel. In an exemplary configuration, the polished alkyl ester stream combined with methanol may pass through the plurality of resin beds at a rate of 0.375 bed volumes per hour. In other configurations, depending on the flow rate and volume of the polished alkyl ester, the rate at which the polished alkyl ester passes through the plurality of resin beds may be more or less than 0.375 bed volumes per hour. The effluent from the plurality of resin beds may then be passed through a reboiler/stripping column to recover alcohol and then treated with an antioxidant. The result product is a refined biodiesel.

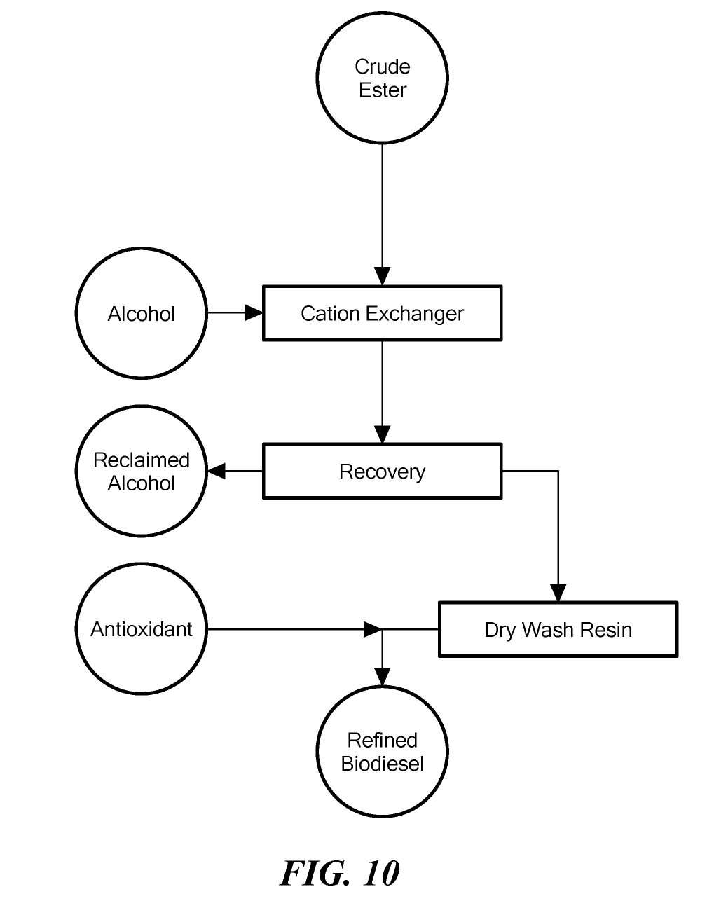

Referring now to FIG. 10, in configurations in which the crude alkyl ester stream contains approximately less than 15 ppm sulfur, for example, in low sulfur feedstocks, the polishing phase may optionally be bypassed, and the crude alkyl ester stream may be introduced or otherwise directed into the cat-ion exchanger to esterify residual PIA in the presence of alcohol. Any excess alcohol may be removed before the alky ester stream enters one or more dry wash adsorption beds, which may include, for example, a resin catalyst. As with high sulfur feedstock processing, an antioxidant may be added to the alkyl ester stream to create refined biodiesel.

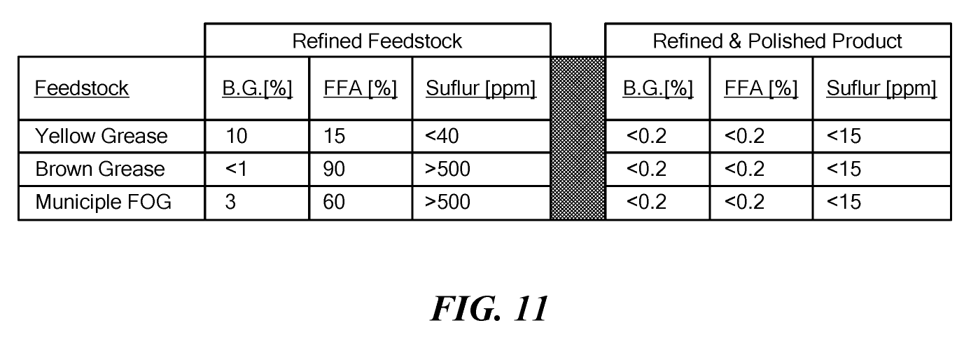

Referring now to FIG. 11, current allowable sulfur concentration for ultra-low-sulfur diesel mandated by the EPA is less than 15 ppm. Analysis of yellow grease, brown grease, and municipal FOG processed into refined biodiesel by the above methods indicates that each of these feedstocks contained less than 15 ppm sulfur and less than 0.2% bound glycerin and free fatty acids.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A method of producing alkyl esters, the method comprising:
pretreating a feedstock including a mixture of glycerides, free fatty acids, and sulfur to remove water and solids to create a pretreated feedstock;
introducing the pretreated feedstock and at least one of an aqueous solution and water into at least one continuous stir tank reactor;
introducing at least one enzyme and alcohol into the at least one continuous stir tank reactor to elicit an enzyme catalyzed reaction with the pretreated feedstock, the enzyme catalyzed reaction creating reacted contents;
separating the reacted contents exiting the at least one continuous stir tank reactor into a glycerin phase and a crude alkyl ester phase;
distilling the crude alkyl ester phase to remove sulfur to provide a polished alkyl ester phase; and
passing the polished alkyl ester phase through a cat-ion exchange apparatus to lower the FFA level producing refined alkyl esters.

2. The method of claim 1, wherein the feedstock is at least one of yellow grease, brown grease, and FOG.

3. The method of claim 1, wherein the distilling the crude alkyl ester includes passing the crude alkyl ester phase through a stripping column in fluid communication with a reboiler.

4. The method of claim 3, wherein the passing the crude alkyl ester phase through the stripping column in fluid communication with a reboiler creates still bottoms, and wherein at least a portion of the still bottoms are diverted into a main still for removal of sulfur.

5. The method of claim 4, wherein the passing the crude alkyl ester phase from the bottoms of the stripping column into the main still further includes introducing the crude alkyl ester phase into to at least one of a curing agent and a physical initiator to promote polymer cross linking

6. The method of claim 4, wherein at least a portion of the still bottoms is diverted from the reboiler loop of the main still for removal of sulfur.

7. The method of claim 1, wherein passing the polished alkyl ester phase through a cat-ion exchange apparatus includes introducing alcohol into the cat-ion exchange apparatus.

8. (canceled)

9. The method of claim 2, wherein the feedstock is recy having a free fatty acid composition of at least 10% by dry weight and at least 50 parts per million sulfur.

10. The method of claim 1, wherein the at least one continuous stir tank reactor includes a plurality of stir tank reactors in series in fluid communication with each other.

11. The method of claim 10, wherein introducing the at least one enzyme and alcohol into the at least one continuous stir tank reactor further includes introducing the at least one enzyme and alcohol into each of the plurality of stir tank reactors.

12. (canceled)

13. The method of claim 1, further including recycling alcohol from the reacted contents before separating the reacted contents into the glycerin phase and the crude alkyl ester phase.

14. The method of claim 1, further including recycling the glycerin phase back into the at least one stirred tank reactor after separating the reacted contents into the glycerin phase and the crude alkyl ester phase.

15. The method of claim 3, further including passing the refined alkyl esters through a second stripping column in fluid communication with a second reboiler.

16. A method of producing alkyl esters, the method comprising:
pretreating a feedstock including a mixture of glycerides, free fatty acids, and sulfur to remove water and solids to create a pretreated feedstock;
introducing the pretreated feedstock and at least one of water and a caustic aqueous solution into a one of a plurality of continuous stir tank reactors arranged in series, the plurality of continuous stir tank reactors being in fluid communication with each other;
introducing at least one enzyme and alcohol into each of the plurality continuous stir tank reactors to elicit an enzyme catalyzed reaction with the pretreated feedstock, the enzyme catalyzed reaction creating reacted contents;
separating the reacted contents exiting the last one in series of the plurality of continuous stir tank reactors into a glycerin phase and a crude alkyl ester phase;
passing the crude alkyl ester phase through a cat-ion exchange apparatus having a plurality of a resin beds to produce refined alkyl esters;
introducing the refined alkyl ester phase into a polar adsorptive media to produce polished alkyl esters; and
treating the polished alkyl esters with an antioxidant.

17. The method of claim 16, further including distilling the refined alkyl esters.

18. The method of claim 17, wherein the distilling the refined alkyl ester includes passing the crude alkyl ester phase through a stripping column in fluid communication with a reboiler.

19. The method of claim 17, further introducing alcohol into the cat-ion exchange apparatus when the crude alkyl ester phase is passed through the cat-ion exchange apparatus.

20. The method of claim 16, wherein the polar adsorptive media is a cat-ion exchange apparatus includes a plurality of resin beds arranged in at least one of in series and in parallel.

21. (canceled)

22. (canceled)

23. (canceled)

24. The method of claim 16, wherein the cat-ion exchange apparatus includes a plurality of resin beds arranged in at least one of in series and in parallel.

25. A method of producing alkyl esters, the method comprising:
pretreating a feedstock having a free fatty acid composition of at least 50% by dry weight and at least 400 parts per million sulfur to remove water and solids to create a pretreated feedstock;
introducing the pretreated feedstock and at least one of water and an aqueous solution into a one of a plurality of continuous stir tank reactors arranged in series, the plurality of continuous stir tank reactors being in fluid communication with each other;
introducing at least one enzyme and alcohol into each of the plurality of continuous stir tank reactors to elicit an enzyme catalyzed reaction with the pretreated feedstock, the enzyme catalyzed reaction creating reacted contents;
separating the reacted contents exiting the at least one continuous stir tank reactor into a glycerin phase and a crude alkyl ester phase;
passing the crude alkyl ester phase through a stripping column in fluid communication with a reboiler to distill the crude alkyl ester phase at a first temperature and to create still bottoms;
diverting at least a portion of the still bottoms into a main still for removal to distill the still bottoms at a second temperature higher than the first temperature to create a polished alkyl ester phase;
passing the polished alkyl ester phase through a plurality of resin beds and introducing alcohol into the plurality of resin beds when the polished alkyl ester phase is passed through the plurality of resin beds to produce refined alkyl esters; and
treating the refined alkyl esters with an antioxidant.

26. (canceled)
